(19) United States
(12) Patent Application Publication
(10) Pub. No.: US 2025/0251536 A1
(43) Pub. Date: Aug. 7, 2025

(54) STEREOSCOPIC SCANNING DEVICE AND STEREOSCOPIC SCANNING METHOD

(71) Applicant: QISDA CORPORATION, Taoyuan City (TW)

(72) Inventors: Chih-Ming Hu, Taoyuan City (TW); Ying-Fang Lin, Taoyuan City (TW); Cheng-Chieh Juan, New Taipei City (TW)

(73) Assignee: QISDA CORPORATION, Taoyuan City (TW)

(21) Appl. No.: 19/012,834

(22) Filed: Jan. 7, 2025

(30) Foreign Application Priority Data
Feb. 7, 2024 (CN) 202410172418.4

(51) Int. Cl. G02B 5/20; A61B 5/00; A61C 9/00; H04N 13/207; H04N 13/254; H04N 23/56; H04N 23/74

(52) U.S. Cl. CPC G02B 5/208; A61B 5/0088; A61C 9/006; H04N 13/207; H04N 13/254; H04N 23/56; H04N 23/74

(57) ABSTRACT

A stereoscopic scanning device is applied by a stereoscopic scanning method and includes a projection module and an imaging module. The projection module includes a structural light generator, a color filter aperture and a visible light source. The structural light generator creates a structured light pattern. The color filter aperture has a first penetration region and a second penetration region, and the first penetration region is smaller than the second penetration region. The visible light source emits a visible beam to the structural light generator, and the structured light pattern passes through the first penetration region to project onto a target object, and the visible beam that does not belong to the structured light pattern passes through the second penetration region to project onto the target object. The imaging module includes an optical sensor used to receive the structure light pattern reflected from the target object.

### BACKGROUND OF THE INVENTION

### 1. Field of the Invention

The present invention relates to a stereoscopic scanning device and a stereoscopic scanning method, and more particularly, to a stereoscopic scanning device with a plaque detection function and a related stereoscopic scanning method.

### 2. Description of the Prior Art

A conventional oral scanner utilizes the visible beam light source and the structural light generator to project the structural light pattern onto the tooth, and further utilizes the optical sensor to receive the structural light pattern reflected from the tooth for acquiring the surface outline of the tooth. The visible beam light source includes the blue light source, the green light source and the red light source. The optical sensor of the conventional oral scanner performs color reconstruction according to the three primary color reflection spectra of the tooth, and true color of the tooth surface outline is displayed on the display screen connected to the oral scanner. However, the conventional oral scanner only has the detection function of the tooth surface outline, and cannot detect the tooth surface outline and further inspect the unhealthy tooth (such as dental plaque or caries) at the same time; in addition, the wavelength range of the red light and the green light are longer than the wavelength range of the blue light, so the green light source and the red light source of the conventional oral scanner is distant from the structural light generator than the blue light source, which results in poor projection efficiency. Design of an oral stereoscopic scanning device capable of detecting the tooth surface outline and checking whether the tooth is decayed is an important issued in the related medical equipment.

### SUMMARY OF THE INVENTION

The present invention provides a stereoscopic scanning device with a plaque detection function and a related stereoscopic scanning method for solving above drawbacks.

According to the claimed invention, a stereoscopic scanning device has a plaque detection function and is applied to a target object. The stereoscopic scanning device includes a projection module and an imaging module. The projection module includes a structural light generator, a color filter aperture and a visible light source. The structural light generator is adapted to generate a structured light pattern. The color filter aperture is disposed on a side of the structural light generator facing the target object. The color filter aperture has a first penetration region and a second penetration region, and a size of the first penetration region is smaller than a size of the second penetration region. The visible light source is adapted to emit visible beams of multiple wavelengths to the structural light generator. The visible beam of a first wavelength is emitted towards the structural light generator to generate the structured light pattern. The structured light pattern passes through the first penetration region to reach the target object, and the visible beam of a second wavelength passes through the second penetration region to reach the target object. The imaging module is relatively disposed adjacent to the projection module. The imaging module includes an optical sensor adapted to receive the structure light pattern reflected from the target object.

According to the claimed invention, a stereoscopic scanning method includes utilizing a visible light source to emit visible beam of multiple wavelengths to a structural light generator. The visible beam of a first wavelength is emitted towards the structural light generator to generate a structured light pattern, the structured light pattern passes through a first penetration region of a color filter to reach a target object, and the visible beam of a second wavelength passes through a second penetration region of a color filter to reach the target object. The stereoscopic scanning method further includes utilizing an optical sensor to receive the structure light pattern reflected from the target object for acquiring structure information of the target object, and utilizing an invisible light source to emit an invisible beam passing through the first penetration region and the second penetration region of the color filter to reach the target object. A size of the first penetration region is smaller than a size of the second penetration region. The stereoscopic scanning method further includes utilizing the optical sensor to receive at least one excitation beam generated by the target object for acquiring lesion information of the target object, and combining the structure information with the lesion information to generate a scanning result. The visible beam of other wavelengths that does not belong to the structured light pattern passes through the second penetration region.

The stereoscopic scanning device and the stereoscopic scanning method of the present invention can dispose the color filter aperture on the projection path; the first penetration region of the color filter aperture can have the small size and allow penetration of the visible beam and the invisible beam, and the second penetration region of the color filter aperture can have the large size and block transmission of the structured light pattern but allow penetration of the invisible beam and the visible beam of other wavelengths that does not belong to the structured light pattern. The optical sensor can be the color sensor. The present invention can respectively activate the blue light emitter of the visible light source and the invisible light source at the same point of time, or alternately activate the blue light emitter of the visible light source and the invisible light source at different points of time. As long as the activated point of time of the invisible light source is different form each activated point of time of the green light emitter and the red light emitter of the visible light source, the present invention can avoid the green fluorescent and the red fluorescent excited by the invisible beam projected onto the target object from being affected by the green light beam and the red light beam of the visible beam. Therefore, the stereoscopic scanning device of the present invention can have a plaque detection function, and can provide the structure information of the colorful tooth model in the normal mode and further provide the lesion information of detecting whether the tooth is the decayed tooth or has the structural defect in the caries mode. Besides, the color image of the tooth model acquired by the stereoscopic scanning device of the present invention can have the preferred depth of field and the preferred projection efficiency.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram of a stereoscopic scanning device according to a first embodiment of the present invention.

FIG. 2 is an appearance side view of a color filter aperture according to the embodiment of the present invention.

FIG. 3 is a diagram of the stereoscopic scanning device according to second embodiment of the present invention.

FIG. 4 is an appearance diagram of the color filter aperture according to the embodiment of the present invention.

FIG. 5 is an appearance diagram of the color filter aperture according to another embodiment of the present invention.

FIG. 6 is a flow chart of a stereoscopic scanning method in the normal mode according to the embodiment of the present invention.

FIG. 7 is a flow chart of the stereoscopic scanning method in the caries mode according to the embodiment of the present invention.

### DETAILED DESCRIPTION

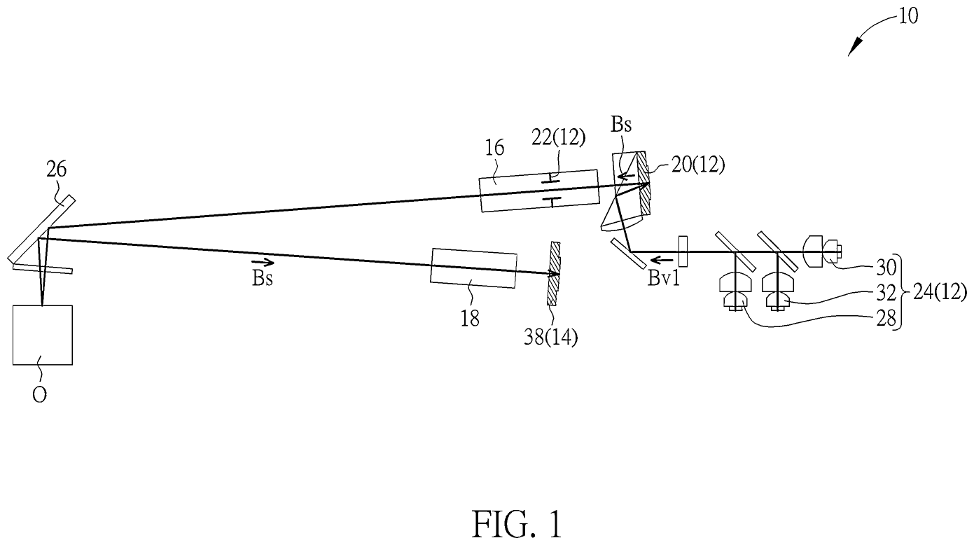

Please refer to FIG. 1. FIG. 1 is a diagram of a stereoscopic scanning device 10 according to a first embodiment of the present invention. The stereoscopic scanning device 10 can include a projection module 12, an imaging module 14, projection lens assembly 16 and imaging lens assembly 18. The stereoscopic scanning device 10 can be an oral scanner or any related medical apparatus. The projection module 12 can provide illumination required for the oral scanner operated in a normal mode and a caries mode. The imaging module 14 can acquire a scanning result of the stereoscopic scanning device 10 in the normal mode and the caries mode. The stereoscopic scanning device 10 set in the normal mode can quickly provide a tooth model. The stereoscopic scanning device 10 set in the caries mode can detect or inspect whether the tooth is a decayed tooth or has a structural defect. The projection lens assembly 16 can be located between the projection module 12 and the target object O. The imaging lens assembly 18 can be located between the imaging module 14 and the target object O. Each of the projection lens assembly 16 and the imaging lens assembly 18 can be composed of several optical elements, and a detailed description is omitted herein for simplicity.

The projection module 12 can include a structural light generator 20, a color filter aperture 22 and a visible light source 24. The visible light source 24 can emit a visible beam Bv1 towards the structural light generator 20, and a structured light pattern Bs generated by the structural light generator 20 can be projected onto the target object O via a reflector 26. The structural light generator 20 can be a digital micromirror device, or any element with a similar function. The color filter aperture 22 can be disposed on a side of the structural light generator 20 facing the target object O. The visible light source 24 can at least include a blue light emitter 28 used to emit a blue light beam towards the structural light generator 20, and can further optionally include a green light emitter 30 and a red light emitter 32 used to respectively emit a green light beam and a red light beam. The visible light source 24 can only utilize the blue light emitter 28 to emit the blue light beam for being the visible beam Bv1, so as to form a blue detection image about of the tooth model; the visible light source 24 may utilize the blue light emitter 28 and the green light emitter 30 and the red light emitter 32 to respectively emit the visible beam Bv1 containing three primary colors, so as to form a colorful detection image about of the tooth model.

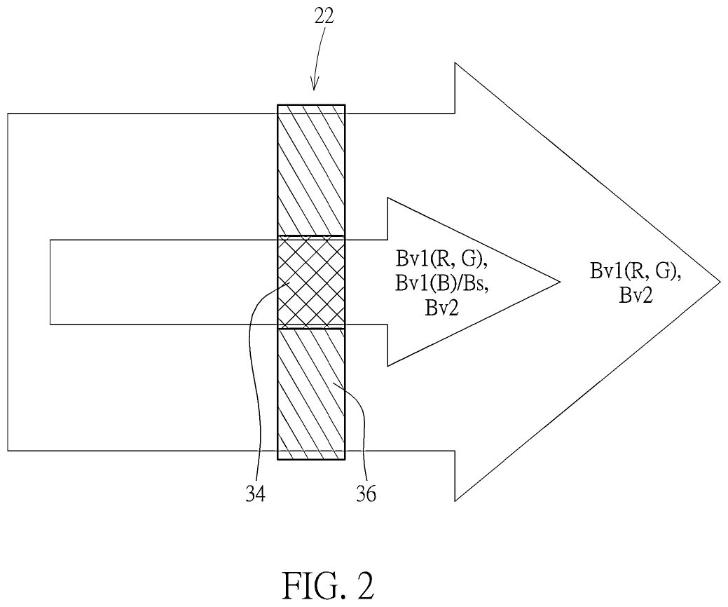

Please refer to FIG. 2. FIG. 2 is an appearance side view of the color filter aperture 22 according to the embodiment of the present invention. The color filter aperture 22 can have a first penetration region 34 and a second penetration region 36. The first penetration region 34 can be shown as a plaid area, the second penetration region 36 can be shown as a twill area covering the plaid area, and a size of the first penetration region 34 can be smaller than a size of the second penetration region 36. The first penetration region 34 can be partly or completely overlapped with the second penetration region 36. The first penetration region 34 can allow penetration of all color light beams. The second penetration region 36 can block transmission of the blue light beam (which means a wavelength range of the structured light pattern Bs), but allow penetration of the light beams of other wavelengths. Therefore, on a projection path between the structural light generator 20 and the reflector 26, the structured light pattern Bs generated by the structural light generator 20 can belong to a blue light band, and the structured light pattern Bs can only pass through the first penetration region 34 to reach the target object O; the visible beam Bv1 of other wavelengths that does not belong to the structured light pattern Bs can pass through the second penetration region 36 to reach the target object O.

The imaging module 14 can be disposed relative to the projection module 12. An optical sensor 38 of the imaging module 14 can receive the structured light pattern Bs and the visible beam Bv1 reflected from the target object O, so as to form the colorful detection image about the tooth model; the optical sensor 38 can be preferably set as a color sensor, which depends on an actual demand. The stereoscopic scanning device 10 can apply the blue light beam for the structured light pattern Bs; practical application of color of the structured light pattern Bs is not limited to the foresaid embodiment. The blue light emitter 28 can be located closer to the structural light generator 20, and the green light emitter 30 and the red light emitter 32 which have longer wavelength range can be located farther away from the structural light generator 20 and therefore have lower projection efficiency. Thus, the present invention can design the color filter aperture 22 with two apertures of different sizes. The first penetration region 34 of the color filter aperture 22 can be a small aperture, and the structured light pattern Bs can pass through the small aperture for maintaining a depth of field of the detection image. The second penetration region 36 of the color filter aperture 22 can be a large aperture where through other primary color light (such as the green light beam and the red light beam) can pass, so as to increase the projection efficiency of the visible beam Bv1 of other wavelengths that does not belong to the structured light pattern Bs.

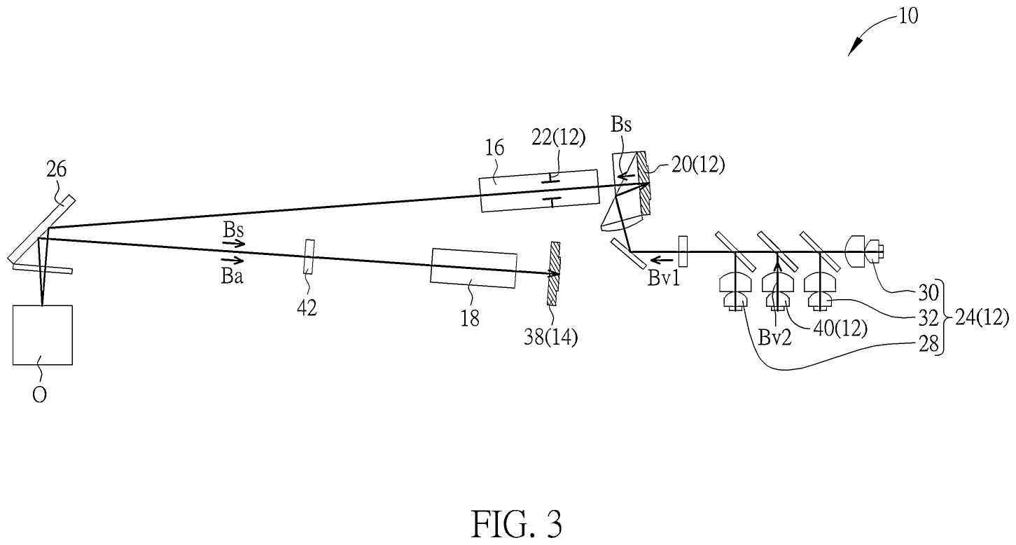

The first embodiment can apply the color filter aperture 22 for the stereoscopic scanning device 10 having the normal mode; however, the color filter aperture 22 can be further applied for the stereoscopic scanning device 10 having the normal mode and the caries mode. Please refer to FIG. 2 and FIG. 3. FIG. 3 is a diagram of the stereoscopic scanning device 10 according to second embodiment of the present invention. In the second embodiment, elements having the same numerals as ones of the first embodiment have the same structures and functions, and a detailed description is omitted herein for simplicity. The projection module 12 of the second embodiment can further include an invisible light source 40, which can be set in the same alignment module as the visible light source 24, and the invisible beam Bv2 emitted by the invisible light source 40 can pass through the projection path where on the color filter aperture 22 is located. The light beam of other wavelength that does not belong to the structured light pattern Bs can pass through the second penetration region 36 of the color filter aperture 22, and the invisible beam Bv2 can pass through the second penetration region 36 to reach the target object O for generating an excitation beam Ba accordingly. The optical sensor 38 can receive the excitation beam Ba excited by the target object O through the imaging path of the imaging lens assembly 18.

In the second embodiment, the first penetration region 34 of the color filter aperture 22 can allow penetration of the visible beam Bv1 and the invisible beam Bv2, the second penetration region 36 can block transmission of the structured light pattern Bs but allow penetration of the invisible beam Bv2 and the visible beam Bv1 of other wavelengths that does not belong to the structured light pattern Bs. As mentioned above, the structured light pattern Bs can belong to the blue light band; the invisible beam Bv2 can be a near ultraviolet light beam, which has a central wavelength about 405 nm. When the target object O is the tooth and the tooth has bacteria (or dental plaque), the invisible beam Bv2 can be projected onto the caries to excite the excitation beam Ba of red fluorescent. If the tooth does not have the bacteria, the invisible beam Bv2 can be projected onto a normal area of the target object O to excite the excitation beam Ba of green fluorescent. If the tooth is broken or in the early stage of the decayed tooth, fluorescence on the target object O is disappeared, and the imaging module 14 can have an image with a partial black pattern on the tooth when the invisible beam Bv2 is projected onto the foresaid tooth; which means the tooth model may have the partial black pattern.

The projection lens assembly 16 can be disposed on a side of the structural light generator 20 facing the target object O. The structured light pattern Bs and the invisible beam Bv2 can be projected onto the target object O through the projection path of the projection lens assembly 16. The imaging lens assembly 18 can be disposed on a side of the structural light generator 20 facing the target object O. The optical sensor 38 can receive the visible beam Bv1 and the invisible beam Bv2 through the imaging path of the imaging lens assembly 18. In addition, the imaging module 14 can further include an invisible light filter 42 disposed on the side of the structural light generator 20 facing the target object O. The invisible light filter 42 can be used to filter the invisible beam Bv2. When the stereoscopic scanning device 10 is switched into the caries mode and the projection module 12 utilizes the invisible light source 40 to emit the invisible beam Bv2, the invisible beam Bv2 projected onto the target object O (such as the tooth) can be excited to generate the excitation beam Ba, and some of the invisible beam Bv2 may be reflected from the target object O to enter the imaging path, so that the invisible light filter 42 can filter the invisible beam Bv2 reflected from the target object O to avoid affecting the detection result of the caries mode.

When the stereoscopic scanning device 10 is switched into the normal mode, the projection module 12 drives the visible light source 24 and the structural light generator 20 to generate the structured light pattern Bs via the blue light beam. The structured light pattern Bs can pass through the first penetration region 34 of the color filter aperture 22 and then be reflected by the reflector 26 towards the target object O, and further be reflected by the target object O for being received by the optical sensor 38, so as to form the blue detection image about the tooth model. Then, the projection module 12 can drive the green light emitter 30 and the red light emitter 32 of the visible light source 24 to respectively emit the visible beam Bv1 belonging to the green light beam and the red light beam. The visible beam Bv1 (such as the green light beam and the red light beam) can pass through the second penetration region 36 of the color filter aperture 22 to project onto the target object O via reflection of the reflector 26, and then be reflected by the target object O for being received by the optical sensor 38; the related detection images can be combined with the blue detection image to form the colorful detection image about the tooth model that has the preferred depth of field and the preferred projection efficiency. It should be mentioned that the visible light source 24 can alternately emit the blue light beam, the green light beam and the red light beam at different points of time.

When the stereoscopic scanning device 10 is switched into the caries mode, the projection module 12 can drive the visible light source 24 and the structural light generator 20 to generate the structured light pattern Bs via the blue light beam. The structured light pattern Bs can pass through the first penetration region 34 of the color filter aperture 22 to project onto the target object O via the reflection of the reflector 26, and then be reflected by the target object O for being received by the optical sensor 38, so as to acquire structure information of the tooth model (which can be the blue detection image). Then, the projection module 12 can drive the invisible light source 40 to emit the invisible beam Bv2. When the invisible beam Bv2 passes through the second penetration region 36 of the color filter aperture 22 to project onto the target object O, the excitation beam Ba of the red fluorescent or the green fluorescent can be excited in accordance with whether the target object O has the bacteria or the structural defect. The optical sensor 38 can receive the excitation beam Ba, and the detection image having lesion information of the decayed tooth or the defected tooth can be combined with the blue detection image to form the colorful detection image about the tooth model that has the preferred depth of field and the preferred projection efficiency.

If the stereoscopic scanning device 10 in the caries mode only drives the blue light emitter 28 of the visible light source 24 to emit the visible beam Bv1 belonging to the blue light beam, the stereoscopic scanning device 10 can drive the visible light source 24 and the invisible light source 40 to respectively emit the visible beam Bv1 (which can be the blue light beam) and the invisible beam Bv2 at the same point of time, or drive the visible light source 24 and the invisible light source 40 to alternately emit the visible beam Bv1 (which can be the blue light beam) and the invisible beam Bv2 at different points of time; the detection result of the decayed tooth or the defected tooth generated by the excitation beam Ba of the red fluorescent or the green fluorescent cannot be interfered by the detection result of the tooth model generated by the structured light pattern Bs that belongs to the blue light beam. If the stereoscopic scanning device 10 in the caries mode drives the green light emitter 30 and the red light emitter 32 of the visible light source 24 to emit the visible beam Bv1 belonging to the green light beam and the red light beam, an activated point of time of the invisible light source 40 can be different from each activated point of time of the green light emitter 30 and the red light emitter 32, so as to avoid the detection result of the decayed tooth or the defected tooth generated by the excitation beam Ba of the red fluorescent or the green fluorescent from being affected by the detection result of the tooth model generated by the visible beam Bv1 belonging to the green light beam and the red light beam.

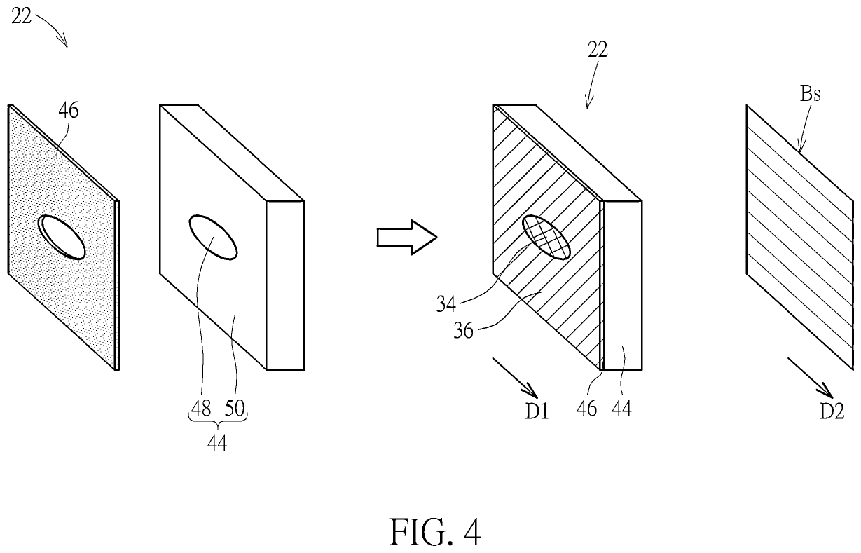

Please refer to FIG. 2 and FIG. 4. FIG. 4 is an appearance diagram of the color filter aperture 22 according to the embodiment of the present invention. The color filter aperture 22 can include a transparent substrate 44 and a light filtering layer. The light filtering layer can be used to absorb or reflect the structured light pattern Bs, so as to block transmission of the structured light pattern Bs. The light filtering layer can be a wavelength absorbing layer 46. The transparent substrate 44 can include an aperture region 48 and an excluded region 50. The wavelength absorbing layer 46 can be used to block transmission of the structured light pattern Bs, and allow penetration of the invisible beam Bv2 and the visible beam Bv1 of other wavelengths that does not belong to the structured light pattern Bs. The aperture region 48 can misalign with (or is not covered by) the wavelength absorbing layer 46 and be set as the first penetration region 34 of the color filter aperture 22. The excluded region 50 can be covered by the wavelength absorbing layer 46 and set as the second penetration region 36 of the color filter aperture 22 with the aperture region 48. The aperture region 48 can be preferably designed as an oval shape, and a long axis D1 of the oval shape can be parallel to a stripe extension direction D2 of the structured light pattern Bs for the preferred depth of field.

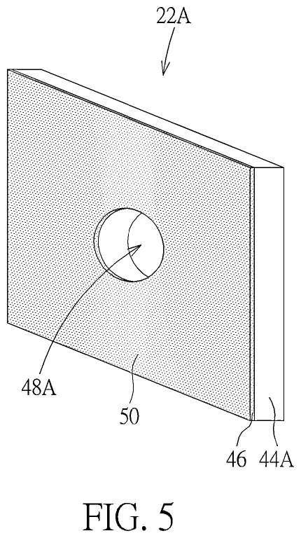

The embodiment shown in FIG. 4 can set a specific range on the transparent substrate 44 as the aperture region 48, and other range of the transparent substrate 44 except the specific range can be set as the excluded region 50, which means the aperture region 48 can be a solid structure of the transparent substrate 44; practical application of the transparent substrate 44 is not limited to the foresaid embodiment. Please refer to FIG. 5. FIG. 5 is an appearance diagram of the color filter aperture 22A according to another embodiment of the present invention. In the embodiment, elements having the same numerals as ones of the foresaid embodiment have the same structures and functions, and the detailed description is omitted here for simplicity. The aperture region 48A of the color filter aperture 22A can be designed as a perforated structure of the transparent substrate 44A, which cannot be covered by the wavelength absorbing layer 46, and can be set as the first penetration region 34 of the color filter aperture 22. The excluded region 50 of the color filter aperture 22A can be covered by the wavelength absorbing layer 46 and set as the second penetration region 36 of the color filter aperture 22 with the aperture region 48A.

Please refer to FIG. 1 and FIG. 3. The embodiments shown in FIG. 1 and FIG. 3 can dispose the green light emitter 30 and the red light emitter 32 on different positions, which depend on a design demand. For example, one possible embodiment may integrate the green light emitter 30 and the red light emitter 32 into the same light channel (which is not shown in the figures), for size reduction of the alignment module of the visible light source 24. Further, the visible light source 24 of the present invention can only include the blue light emitter 28 and a yellow light emitter (which is not shown in the figures) used to emit the visible beam of a first wavelength (such as the blue light beam) and the visible beam of a second wavelength (such as a yellow light beam). The yellow light beam can be decomposed into the green light beam and the red light beam, and other embodiment of the present invention can dispose a light filtering element (which is not shown in the figures) accordingly on the projection path of the yellow light beam emitted by the yellow light emitter, so the optical sensor 38 can receive the three primary color light beam to acquire the colorful detection image. In addition, the stereoscopic scanning device 10 may further set an infrared light source (which is not shown in the figures) as the invisible light source 40, which can emit infrared light used to penetrate biological tissue for detecting the decayed tooth or the structural defect, and the detailed description is omitted herein for simplicity.

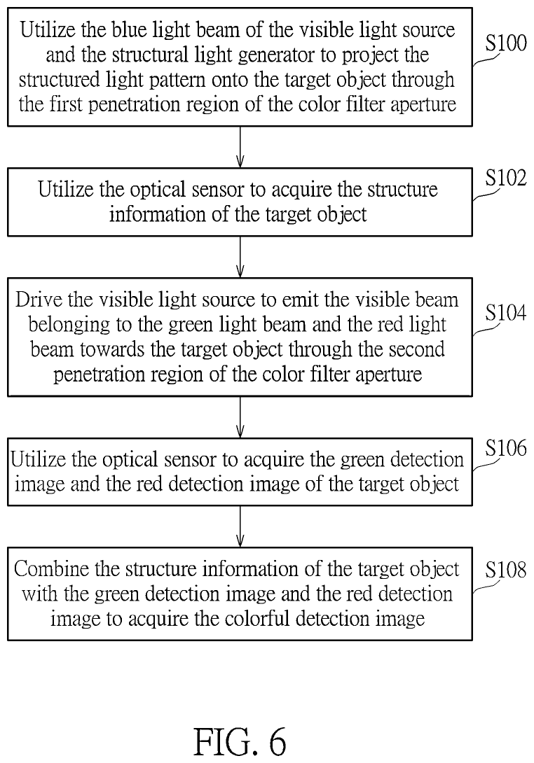

Please refer to FIG. 6. FIG. 6 is a flow chart of a stereoscopic scanning method in the normal mode according to the embodiment of the present invention. When the stereoscopic scanning device 10 is switched into the normal mode, step S100 can be executed to utilize the blue light beam of the visible light source 24 and the structural light generator 20 to project the structured light pattern Bs onto the target object O. The structured light pattern Bs can pass through the first penetration region 34 of the color filter aperture 22, and be blocked by the second penetration region 36. Then, step S102 can be executed to utilize the optical sensor 38 to receive the structured light pattern Bs that is reflected by the target object O and passes through the imaging lens assembly 18, for acquiring the structure information of the target object O. Then, step S104 can be executed to utilize the visible light source 24 to emit the visible beam Bv1 belonging to the green light beam and the red light beam; the green light beam and the red light beam can pass through the second penetration region 36 of the color filter aperture 22. Step S106 can be executed to utilize the optical sensor 38 to acquire the green detection image and the red detection image of the target object O. Final, step S108 can be executed to combine the structure information of the target object O (that belongs to the blue detection image) with the green detection image and the red detection image, for acquiring the final colorful detection image. In the embodiment, the visible light source 24 can alternately emit the blue light beam, the green light beam and the red light beam at different points of time.

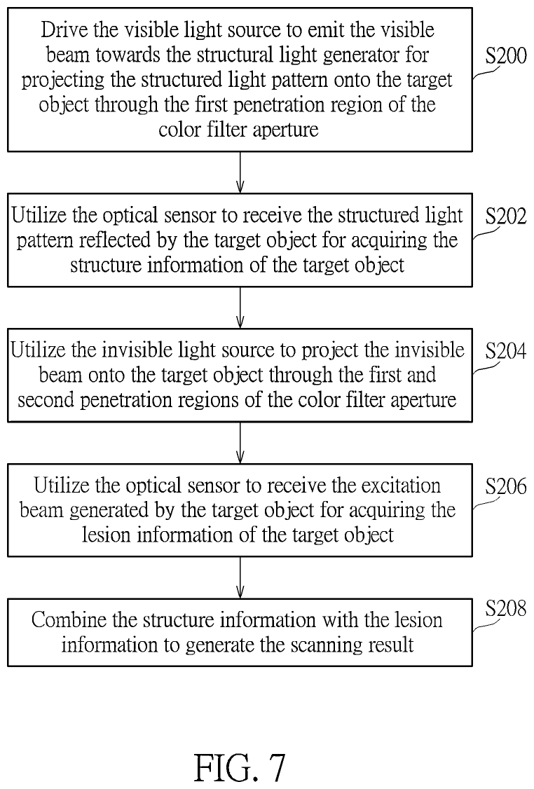

Please refer to FIG. 7. FIG. 7 is a flow chart of the stereoscopic scanning method in the caries mode according to the embodiment of the present invention. When the stereoscopic scanning device 10 is switched into the caries mode, step S200 can be executed to utilize the blue light beam of the visible light source 24 and the structural light generator 20 to project the blue light pattern of the structured light pattern Bs onto the target object O; the color filter aperture 22 can be disposed between the structural light generator 20 and the target object O, so the structured light pattern Bs can pass through the first penetration region 34 of the color filter aperture 22 to reach the target object O, but be blocked by the second penetration region 36 of the color filter aperture 22. If the visible beam Bv1 has other color light, the visible beam Bv1 of other wavelengths that does not belong to the structured light pattern Bs can pass through the second penetration region 36 of the color filter aperture 22 to reach the target object O. Then, step S202 can be executed to utilize the optical sensor 38 to receive the structured light pattern Bs reflected by the target object O for acquiring the structure information of the target object O. Then, step S204 can be executed to utilize the invisible light source 40 to project the invisible beam Bv2 onto the target object O; in the meantime, the invisible beam Bv2 can pass through the second penetration region 36 of the color filter aperture 22. Final, step S206 and step S208 can be executed to utilize the optical sensor 38 to receive the excitation beam Ba generated by the target object O for acquiring the lesion information of the target object O, and combine the structure information with the lesion information to generate the scanning result.

In step S200, the structured light pattern Bs (which is the blue light pattern) can be blocked by the second penetration region 36 of the color filter aperture 22, and only pass through the first penetration region 34 of the color filter aperture 22, so that the structure information of the target object O acquired by the optical sensor 38 in step S202 can have the preferred depth of field. In addition, if other color light exists in step S200, the visible beam Bv1 of other wavelengths that does not belong to the structured light pattern Bs can pass through the second penetration region 36 of the color filter aperture 22, and the invisible beam Bv2 in step S204 can pass through the second penetration region 36 of the color filter aperture 22, so the projection efficiency of other primary color light (which means the green light and the red light) can be increased accordingly, and the optical sensor 38 set by the color sensor can be preferably used to acquire the structure information of the colorful tooth model or the lesion information detected by the excitation beam Ba.

In conclusion, the stereoscopic scanning device and the stereoscopic scanning method of the present invention can dispose the color filter aperture on the projection path; the first penetration region of the color filter aperture can have the small size and allow penetration of the visible beam and the invisible beam, and the second penetration region of the color filter aperture can have the large size and block transmission of the structured light pattern but allow penetration of the invisible beam and the visible beam of other wavelengths that does not belong to the structured light pattern. The optical sensor can be the color sensor. The present invention can respectively activate the blue light emitter of the visible light source and the invisible light source at the same point of time, or alternately activate the blue light emitter of the visible light source and the invisible light source at different points of time. As long as the activated point of time of the invisible light source is different form each activated point of time of the green light emitter and the red light emitter of the visible light source, the present invention can avoid the green fluorescent and the red fluorescent excited by the invisible beam projected onto the target object from being affected by the green light beam and the red light beam of the visible beam. Therefore, the stereoscopic scanning device of the present invention can have a plaque detection function, and can provide the structure information of the colorful tooth model in the normal mode and further provide the lesion information of detecting whether the tooth is the decayed tooth or has the structural defect in the caries mode. Besides, the color image of the tooth model acquired by the stereoscopic scanning device of the present invention can have the preferred depth of field and the preferred projection efficiency.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A stereoscopic scanning device having a plaque detection function and applied to a target object, the stereoscopic scanning device comprising:
a projection module, comprising:
a structural light generator adapted to generate a structured light pattern;
a color filter aperture disposed on a side of the structural light generator facing the target object, the color filter aperture having a first penetration region and a second penetration region, and a size of the first penetration region being smaller than a size of the second penetration region; and
a visible light source adapted to emit visible beams of multiple wavelengths to the structural light generator, the visible beam of a first wavelength being emitted towards the structural light generator to generate the structured light pattern, the structured light pattern passing through the first penetration region to reach the target object, the visible beam of a second wavelength passing through the second penetration region to reach the target object; and
an imaging module relatively disposed adjacent to the projection module, the imaging module comprising:
an optical sensor adapted to receive the structure light pattern reflected from the target object.

2. The stereoscopic scanning device of claim 1, wherein the projection module further comprises an invisible light source adapted to emit an invisible beam passing through the first penetration region and the second penetration region to reach the target object and further to generate at least one excitation beam, the optical sensor is adapted to further receive the excitation beam generated by the target object.

3. The stereoscopic scanning device of claim 2, wherein the first penetration region allows penetration of the visible beam and the invisible beam, the second penetration region blocks transmission of the structured light pattern but allows penetration of the invisible beam and the visible beam of the second wavelength.

4. The stereoscopic scanning device of claim 1, wherein the first penetration region is partly or completely overlapped with the second penetration region.

5. The stereoscopic scanning device of claim 1, wherein the color filter aperture comprises a transparent substrate and a light filtering layer, the transparent substrate comprises an aperture region and an excluded region, the aperture region is a solid structure or a perforated structure of the transparent substrate, the light filtering layer is adapted to block the structured light pattern, the aperture region misaligns with the light filtering layer and is set as the first penetration region, the excluded region is covered by the light filtering layer and set as the second penetration region with the aperture region.

6. The stereoscopic scanning device of claim 5, wherein the aperture region is an oval shape, a long axis of the oval shape is parallel to a stripe extension direction of the structured light pattern.

7. The stereoscopic scanning device of claim 2, wherein the visible beam is a blue light beam, the invisible beam is a near ultraviolet light beam, the excitation beam is red fluorescent and/or green fluorescent, the visible light source and the invisible light source are adapted to respectively emit the visible beam and the invisible beam at the same point of time, or to alternately emit the visible beam and the invisible beam at different points of time.

8. The stereoscopic scanning device of claim 2, wherein the visible light source further comprises a blue light emitter, a green light emitter and a red light emitter, adapted to alternately emit the visible beam belonging to a blue light beam, a green light beam and a red light beam at different points of time, an activated point of time of the invisible light source is different from each activated point of time of the green light emitter and the red light emitter.

9. The stereoscopic scanning device of claim 2, wherein the visible light source further comprises a blue light emitter and a yellow light emitter, the invisible light source is an infrared light source.

10. The stereoscopic scanning device of claim 1, wherein the optical sensor is a color sensor.

11. The stereoscopic scanning device of claim 2, wherein the projection module further comprises projection lens assembly disposed on the side of the structural light generator facing the target object, the structured light pattern and the invisible beam are projected onto the target object through the projection lens assembly, the imaging module further comprises imaging lens assembly disposed on a side of the optical sensor facing the target object, the optical sensor receives the visible beam and the invisible beam passing through the imaging lens assembly.

12. The stereoscopic scanning device of claim 1, wherein the imaging module further comprises an invisible light filter disposed on a side of the optical sensor facing the target object, and adapted to filter the invisible beam reflected from the target object.

13. A stereoscopic scanning method, comprising:
utilizing a visible light source to emit visible beam of multiple wavelengths to a structural light generator, wherein the visible beam of a first wavelength is emitted towards the structural light generator to generate a structured light pattern, the structured light pattern passes through a first penetration region of a color filter to reach a target object, and the visible beam of a second wavelength passes through a second penetration region of a color filter to reach the target object;
utilizing an optical sensor to receive the structure light pattern reflected from the target object for acquiring structure information of the target object;
utilizing an invisible light source to emit an invisible beam passing through the first penetration region and the second penetration region of the color filter to reach the target object, wherein a size of the first penetration region is smaller than a size of the second penetration region;
utilizing the optical sensor to receive at least one excitation beam generated by the target object for acquiring lesion information of the target object; and
combining the structure information with the lesion information to generate a scanning result;
wherein the visible beam of other wavelengths that does not belong to the structured light pattern passes through the second penetration region.

14. The stereoscopic scanning method of claim 13, wherein the structured light pattern is a blue light pattern, the invisible beam is a near ultraviolet light beam, the excitation beam is red fluorescent and/or green fluorescent, the stereoscopic scanning method further comprises:
activating the visible light source and the invisible light source respectively at the same point of time, or alternately at different points of time.

15. The stereoscopic scanning method of claim 13, wherein the visible light source further comprises a blue light emitter, a green light emitter and a red light emitter adapted to respectively emit a blue light beam, a green light beam and a red light beam, the stereoscopic scanning method further comprises:
activating the light emitters of the visible light source alternately at different points of time.

16. The stereoscopic scanning method of claim 13, wherein the first penetration region allows penetration of the visible beam and the invisible beam, the second penetration region blocks transmission of the structured light pattern.
